(19) United States
(12) Patent Application Publication
(10) Pub. No.: US 2010/0113507 A1
(43) Pub. Date: May 6, 2010

(54) 3-Tetrazolyl Indazoles, 3-Tetrazolyl Pyrazolopyridines, and use Thereof

(75) Inventors: Chantal Fürstner, Mulheim/Ruhr (DE); Hartmut Schirok, Wuppertal (DE); Nils Griebenow, Dormagen (DE); Joachim Mittendorf, Wuppertal (DE); Johannes-Peter Stasch, Solingen (DE); Frank Wunder, Wuppertal (DE)

Correspondence Address: EDWARDS ANGELL PALMER & DODGE LLP, P.O. BOX 55874, BOSTON, MA, 02205, US

(73) Assignee: BAYER HEALTHCARE AG, Leverkusen (DE)

(21) Appl. No.: 12/299,906

(22) PCT Filed: Apr. 30, 2007
(86) PCT No.: PCT/EP07/03814

§ 371 Date: May 18, 2009

(30) Foreign Application Priority Data
May 9, 2006 (DE) 10 2006 021 733.0

(51) Int. Cl. A61K 31/437; A61P 9/00; A61P 11/00; A61P 25/28; A61P 25/16; A61P 29/00; C07D 471/04

(52) U.S. Cl. 514/303; 546/119

(57) ABSTRACT

The present application relates to novel 3-tetrazolylindazole and 3-tetrazolylpyrazolo[3,4-b]-pyridine derivatives, processes for their preparation, their use alone or in combination for the treatment and/or prophylaxis of diseases, and their use for producing medicaments for the treatment and/or prophylaxis of diseases, especially for the treatment and/or prevention of cardiovascular disorders.

The present application relates to novel 3-tetrazolylindazole and 3-tetrazolylpyrazolo[3,4-b]-pyridine derivatives, processes for their preparation, their use alone or in combinations for the treatment and/or prophylaxis of diseases, and their use for producing medicaments for the treatment and/or prophylaxis of diseases, especially for the treatment and/or prevention of cardiovascular disorders.

One of the most important cellular transmission systems in mammalian cells is cyclic guanosine monophosphate (cGMP). Together with nitric oxide (NO), which is released from the endothelium and transmits hormonal and mechanical signals, it foams the NO/cGMP system. Guanylate cyclases catalyze the biosynthesis of cGMP from guanosine triphosphate (GTP). The representatives of this family disclosed to date can be divided both according to structural features and according to the type of ligands into two groups: the particulate guanylate cyclases which can be stimulated by natriuretic peptides, and the soluble guanylate cyclases which can be stimulated by NO. The soluble guanylate cyclases consist of two subunits and very probably contain one heme per heterodimer, which is part of the regulatory site. The latter is of central importance for the mechanism of activation. NO is able to bind to the iron atom of heme and thus markedly increase the activity of the enzyme. Heme-free preparations cannot, by contrast, be stimulated by NO. Carbon monoxide (CO) is also able to attach to the central iron atom of heme, but the stimulation by CO is distinctly less than that by NO.

Through the production of cGMP and the regulation, resulting therefrom, of phosphodiesterases, ion channels and protein kinases, guanylate cyclase plays a crucial part in various physiological processes, in particular in the relaxation and proliferation of smooth muscle cells, in platelet aggregation and adhesion and in neuronal signal transmission, and in disorders caused by an impairment of the aforementioned processes. Under pathophysiological conditions, the NO/cGMP system may be suppressed, which may lead for example to high blood pressure, platelet activation, increased cellular proliferation, endothelial dysfunction, atherosclerosis, angina pectoris, heart failure, myocardial infarction, thromboses, stroke and sexual dysfunction.

A possible way of treating such disorders which is independent of NO and aims at influencing the cGMP signaling pathway in organisms is a promising approach because of the high efficiency and few side effects which are to be expected.

Compounds, such as organic nitrates, whose effect is based on NO have to date been exclusively used for the therapeutic stimulation of soluble guanylate cyclase. NO is produced by bioconversion and activates soluble guanylate cyclase by attaching to the central iron atom of heme. Besides the side effects, the development of tolerance is one of the crucial disadvantages of this mode of treatment.

Some substances which directly stimulate soluble guanylate cyclase, i.e. without previous release of NO, have been described in recent years, such as, for example, 3-(5′-hydroxymethyl-2′-furyl)-1-benzylindazole [YC-1, Wu et al., Blood 84 (1994), 4226; Mülsch et al., Brit. J. Pharmacol. 120 (1997), 681], fatty acids [Goldberg et al., J. Biol. Chem. 252 (1977), 1279], diphenyliodonium hexafluorophosphate [Pettibone et al., Eur. J. Pharmacol. 116 (1985), 307], isoliquiritigenin [Yu et al., Brit. J. Pharmacol. 114 (1995), 1587] and various substituted pyrazole derivatives (WO 98/16223).

Further fused pyrazole derivatives with heterocyclic substituents are described inter alia in WO 98/16507, WO 98/23619 and WO 00/06569 as stimulators of soluble guanylate cyclase. However, it has emerged that these compounds display disadvantages in relation to their in vivo properties, such as, for example, their behavior in the liver, their pharmacokinetic behavior, their dose-effect relation and/or their metabolic pathway.

A moderately vasorelaxant effect of the compound 1-(2-fluorobenzyl)-3-(1H-tetrazol-5-yl)-1H-pyrazolo[3,4-b]pyridine is reported by A. Straub et al., Bioorg. Med. Chem. Lett. 11, 781-784 (2001). Various 1-benzyl-3-(1H-tetrazol-5-yl)-1H-indazole derivatives are disclosed by G. Corsi et al., J. Med. Chem. 19 (6), 778-783 (1976). In addition, WO 2005/030121 discloses fused pyrazole derivatives with heterocyclic substituents for the treatment of neoplastic diseases. WO 01/57024 claims certain indazoles with heterocyclic substituents and their use for blocking voltage-gated sodium channels in glaucoma and multiple sclerosis.

It was an object of the present invention to provide novel substances which act as stimulators of soluble guanylate cyclase and display an improved activity by comparison with the compounds disclosed in the prior art.

Specifically, the present invention relates to compounds of the general formula (I)

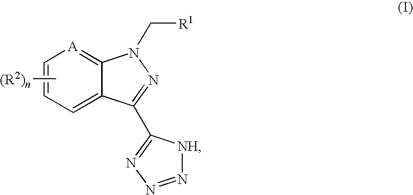

in which
A is CH, CR² or N,
R¹ is phenyl, pyridyl, furyl, thienyl, thiazolyl, oxazolyl, isothiazolyl or isoxazolyl, each of which may be substituted up to twice, identically or differently, by halogen, cyano, (C₁-C₄)-alkyl, trifluoromethyl and/or (C₂-C₄)-alkynyl,
   or
   is (C₅-C₇)-cycloalkyl which may be substituted up to twice, identically or differently, by fluorine and/or (C₁-C₄)-alkyl,
R² is a substituent selected from the series halogen, cyano, (C₁-C₄)-alkyl, trifluoromethyl, amino, (C₁-C₄)-alkoxy and trifluoromethoxy,
   and
n is the number 0, 1 or 2,
   where, in the event that the substituent R² occurs more than once, its meanings may be identical or different,
      and the salts, solvates and solvates of the salts thereof,
      with the exception of the compounds 1-(2-fluorobenzyl)-3-(1H-tetrazol-5-yl)-1H-pyrazolo[3,4-b]pyridine, 1-(4-chlorobenzyl)-3-(1H-tetrazol-5-yl)-1H-indazole, 1-(2,4-dichlorobenzyl)-3-(1H-tetrazol-5-yl)-1H-indazole and 1-(4-chloro-2-methylbenzyl)-3-(1H-tetrazol-5-yl)-1H-indazole.

Compounds according to the invention are the compounds of the formula (I) and the salts, solvates and solvates of the salts thereof, the compounds which are encompassed by formula (I) and are of the formulae mentioned hereinafter, and the salts, solvates and solvates of the salts thereof, and the compounds which are encompassed by formula (I) and are mentioned hereinafter as exemplary embodiments, and the salts, solvates and solvates of the salts thereof, insofar as the compounds encompassed by formula (I) and mentioned hereinafter are not already salts, solvates and solvates of the salts.

The compounds according to the invention may, depending on their structure, exist in stereoisomeric forms (enantiomers, diastereomers). The present invention therefore relates to the enantiomers or diastereomers and respective mixtures thereof. The stereoisomerically pure constituents can be isolated in a known manner from such mixtures of enantiomers and/or diastereomers.

Where the compounds according to the invention can occur in tautomeric forms, the present invention encompasses all tautomeric forms.

Salts preferred for the purposes of the present invention are physiologically acceptable salts of the compounds according to the invention. However, salts which are themselves unsuitable for pharmaceutical applications but can be used for example for isolating or purifying the compounds according to the invention are also encompassed.

Physiologically acceptable salts of the compounds according to the invention include acid addition salts of mineral acids, carboxylic acids and sulfonic acids, e.g. salts of hydrochloric acid, hydrobromic acid, sulfuric acid, phosphoric acid, methanesulfonic acid, ethanesulfonic acid, toluenesulfonic acid, benzenesulfonic acid, naphthalenedisulfonic acid, acetic acid, trifluoroacetic acid, propionic acid, lactic acid, tartaric acid, malic acid, citric acid, fumaric acid, maleic acid and benzoic acid.

Physiologically acceptable salts of the compounds according to the invention also include salts of conventional bases such as, for example and preferably, alkali metal salts (e.g. sodium and potassium salts), alkaline earth metal salts (e.g. calcium and magnesium salts) and ammonium salts derived from ammonia or organic amines having 1 to 16 C atoms, such as, for example and preferably, ethylamine, diethylamine, triethylamine, ethyldiisopropylamine, monoethanolamine, diethanolamine, triethanolamine, dicyclohexylamine, dimethylaminoethanol, procaine, dibenzylamine, N-methyl-morpholine, arginine, lysine, ethylenediamine and N-methylpiperidine.

Solvates refer for the purposes of the invention to those foams of the compounds according to the invention which form a complex in the solid or liquid state through coordination with solvent molecules. Hydrates are a specific form of solvates in which the coordination takes place with water. Solvates preferred in the context of the present invention are hydrates.

The present invention also encompasses prodrugs of the compounds according to the invention. The term “prodrugs” encompasses compounds which themselves may be biologically active or inactive but are converted during their residence time in the body into compounds according to the invention (for example by metabolism or hydrolysis).

In the context of the present invention, the substituents have the following meaning unless otherwise specified:

(C₁-C₄)-Alkyl is in the context of the invention a straight-chain or branched alkyl radical having 1 to 4 carbon atoms. Examples which may be preferably mentioned are: methyl, ethyl, n-propyl, isopropyl, n-butyl, isobutyl, sec-butyl and tert-butyl.

(C₂-C₄)-Alkynyl is in the context of the invention a straight-chain or branched alkynyl radical having 2 to 4 carbon atoms and a triple bond. A straight-chain alkynyl radical having 2 to 4 carbon atoms is preferred. Examples which may be preferably mentioned are: ethynyl, n-prop-1-yn-1-yl, n-prop-2-yn-1-yl, n-but-1-yn-1-yl, n-but-2-yn-1-yl and n-but-3-yn-1-yl.

(C₁-C₄)-Alkoxy is in the context of the invention a straight-chain or branched alkoxy radical having 1 to 4 carbon atoms. Examples which may be preferably mentioned are: methoxy, ethoxy, n-propoxy, isopropoxy, n-butoxy and tert-butoxy.

(C₅-C₇)-Cycloalkyl is in the context of the invention a monocyclic, saturated cycloalkyl group having 5 to 7 ring carbon atoms. Examples which may be preferably mentioned are: cyclopentyl, cyclohexyl and cycloheptyl.

Halogen in the context of the invention includes fluorine, chlorine, bromine and iodine. Fluorine or chlorine are preferred.

If radicals in the compounds according to the invention are substituted, the radicals may, unless otherwise specified, be substituted one or more times. In the context of the present invention, all radicals which occur more than once have a mutually independent meaning. Substitution by one, two or three identical or different substituents is preferred. Substitution by one substituent is very particularly preferred.

Preference is given in the context of the present invention to compounds of the formula (I) in which
A is N,
R¹ is pyridyl, furyl, thienyl, thiazolyl, oxazolyl, isothiazolyl or isoxazolyl, each of which may be substituted up to twice, identically or differently, by fluorine, chlorine, bromine, cyano, methyl and/or trifluoromethyl,
   is (C₅-C₇)-cycloalkyl which may be substituted up to twice, identically or differently, by fluorine and/or methyl,
   or
   is a substituted ortho-fluorophenyl group of the formula

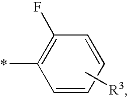

in which
* is the point of linkage
and
R³ is fluorine, chlorine, cyano, methyl or trifluoromethyl,
R² is a substituent selected from the series fluorine, chlorine, methyl, trifluoromethyl, amino, methoxy and trifluoromethoxy,
   and
n is the number 0 or 1,
   and the salts, solvates and solvates of the salts thereof.

Particular preference is given in the context of the present invention to compounds of the formula (I) with the following structures:

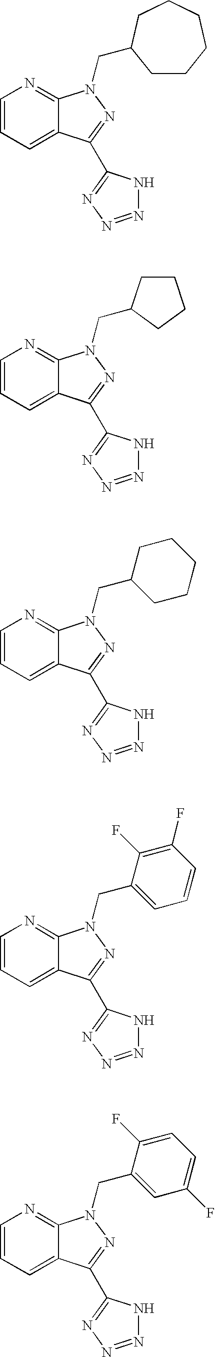

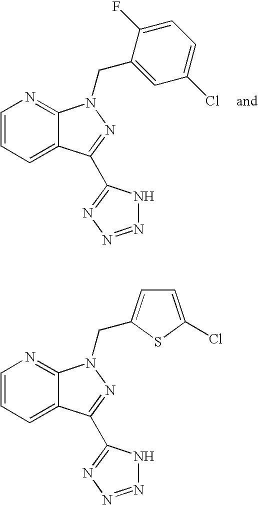

and the salts, solvates and solvates of the salts thereof.

The definitions of radicals indicated specifically in their respective combinations or preferred combinations of radicals are replaced as desired irrespective of the particular combinations indicated for the radicals also by definitions of radicals of other combinations.

Combinations of two or more of the abovementioned preferred ranges are very particularly preferred.

The invention further relates to a process for preparing the compounds of the invention of the formula (I), characterized in that a compound of the formula (II)

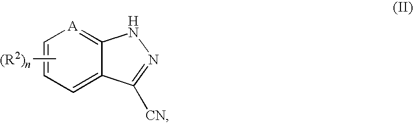

in which A, R² and n each have the meanings indicated above,
is converted in an inert solvent in the presence of a base with a compound of the formula (III)

R¹—CH₂—X (III),

in which
R¹ has the meaning indicated above,
and
X is a leaving group such as halogen, mesylate, tosylate or triflate,
into a compound of the formula (IV)

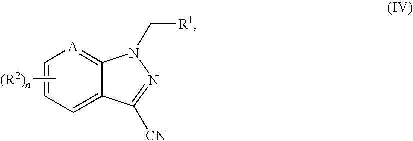

in which A, R¹, R² and n each have the meanings indicated above,
and the latter is then reacted in an inert solvent with an alkali metal azide in the presence of an acid or with trimethylsilyl azide, where appropriate in the presence of a catalyst,
and the compounds of the invention obtained in this way are converted where appropriate with the appropriate (i) solvents and/or (ii) acids or bases into the solvates, salts and/or solvates of the salts thereof.

Examples of inert solvents for process step (II)+(III)→(IV) are ethers such as diethyl ether, methyl tert-butylether, dioxane, tetrahydrofuran, glycol dimethyl ether or diethylene glycol dimethyl ether, hydrocarbons such as benzene, toluene, xylene, hexane, cyclohexane or petroleum fractions, halohydrocarbons such as dichloromethane, trichloromethane, tetrachloromethane, 1,2-dichloroethane, trichloroethane, tetrachloroethane, trichloroethylene, chlorobenzene or chloro-toluene, or other solvents such as dimethylformamide (DMF), dimethyl sulfoxide (DMSO), N,N′ dimethylpropyleneurea (DMPU), N-methylpyrrolidone (NMP), acetone, acetonitrile or pyridine. It is likewise possible to employ mixtures of the solvents mentioned. Dimethylformamide is preferably used.

Customary inorganic or organic bases are suitable as base for process step (II)+(III)→(IV). These include preferably alkali metal hydroxides such as, for example, lithium, sodium or potassium hydroxide, alkali metal or alkaline earth metal carbonates such as lithium, sodium, potassium, calcium or cesium carbonate, alkali metal alcoholates such as sodium or potassium tert-butoxide, alkali metal hydrides such as sodium or potassium hydride, amides such as lithium or potassium bis(trimethylsilyl)amide or lithium diisopropylamide, organometallic compounds such as butyllithium or phenyllithium, or organic amines such as triethylamine, N-methylmorpholine, N-methylpiperidine, N,N-diisopropylethylamine or pyridine. Cesium carbonate is preferably used.

Process step (II)+(III)→(IV) is generally carried out in a temperature range from 0° C. to +100° C., preferably at +20° C. to +50° C. The reaction can take place under atmospheric, elevated or reduced pressure (e.g. from 0.5 to 5 bar). It is generally carried out under atmospheric pressure.

Examples of inert solvents for process step (IV)→(I) are ethers such as diethyl ether, dioxane, tetrahydrofuran, glycol dimethyl ether or diethylene glycol dimethyl ether, hydrocarbons such as benzene, toluene, xylene, hexane, cyclohexane or petroleum fractions, or other solvents such as dimethyl sulfoxide, dimethylformamide, N,N′-dimethylpropyleneurea (DMPU) or N-methyl-pyrrolidone (NMP). It is likewise possible to employ mixtures of the solvents mentioned. Toluene is preferably used.

The suitable azide reagent for this process step is in particular sodium azide in the presence of a proton source such as ammonium chloride or acetic acid, or trimethylsilyl azide. The latter reaction can advantageously be carried out in the presence of a catalyst. Compounds suitable for this purpose are in particular ones such as di-n-butyltin oxide, trimethylaluminum or zinc bromide. Trimethylsilyl azide in combination with di-n-butyltin oxide is preferably used.

Process step (IV)→(I) is generally carried out in a temperature range from +50° C. to +150° C., preferably at +60° C. to +120° C. The reaction can be carried out under atmospheric, elevated or reduced pressure (e.g. from 0.5 to 5 bar). It is generally carried out under atmospheric pressure.

The compounds of the formula (II) are disclosed in the literature or can be prepared in analogy to processes disclosed in the literature [cf., for example, WO 00/06569; G. M. Shutske et al., J. Heterocycl. Chem. 34, 789 (1997); H. Salkowski, Chem. Ber. 17, 506 (1884), ibid., 22, 2139 (1889); M. M. Abdel-Khalik et al., Synthesis, 1166 (2000)]. The compound of the formula (II) in which A is N and n is 0 can also be obtained starting from 2-fluoropyridine (V)

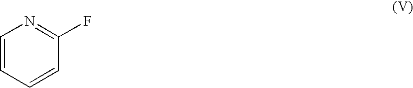

by acylation with ethyl trifluoroacetate and subsequent condensation with hydrazine to give 3-(tri-fluoromethyl)pyrazolopyridine of the formula (VI)

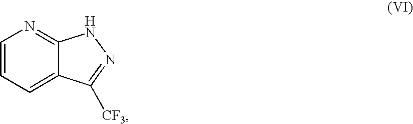

and subsequent reaction of (VI) with ammonia (see reaction scheme below).

The compounds of the formulae (III) and (V) are commercially available, disclosed in the literature or can be prepared in analogy to processes disclosed in the literature.

Preparation of the compounds of the invention can be illustrated by the following synthesis scheme:

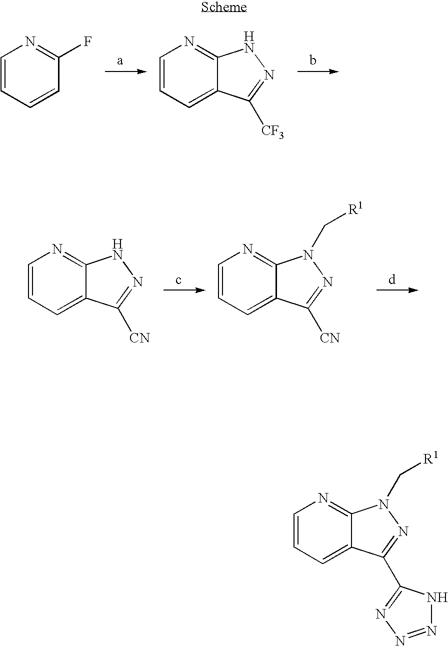

[(a): 1. LDA, THF; 2. CF₃CO₂Et; 3. hydrazine hydrate; (b): aq. NH₃; (c): R¹—CH₂—X, base; (d): Me₃SiN₃, cat. (n-Bu)₂SnO].

The compounds of the invention have valuable pharmacological properties and can be used for the prevention and treatment of disorders in humans and animals.

The compounds of the invention open up a further treatment alternative and represent an enrichment of pharmacy. Compared with the substances disclosed in the prior art, the compounds of the invention surprisingly show a more potent vasorelaxant effect.

The compounds of the invention further inhibit platelet aggregation and lead to a reduction in blood pressure and to an increase in the coronary blood flow. These effects are mediated by a direct stimulation of soluble guanylate cyclase and an intracellular increase in cGMP. In addition, the compounds of the invention enhance the effect of substances which increase the cGMP level, such as, for example, EDRF (endothelium-derived relaxing factor), NO donors, protoporphyrin IX, arachidonic acid or phenylhydrazine derivatives.

The compounds according to the invention can therefore be employed in medicaments for the treatment of cardiovascular disorders such as, for example, for the treatment of high blood pressure and heart failure, stable and unstable angina pectoris, pulmonary hypertension, peripheral and cardiac vascular disorders, arrhythmias, for the treatment of thromboembolic disorders and ischemias such as myocardial infarction, stroke, transistoric and ischemic attacks, disturbances of peripheral blood flow, reperfusion damage, for the prevention of restenoses as after thrombolysis therapies, percutaneous transluminal angioplasties (PTAs), percutaneous transluminal coronary angioplasties (PTCAs) and bypass and for the treatment of arteriosclerosis, asthmatic disorders and diseases of the urogenital system such as, for example, prostate hypertrophy, erectile dysfunction, female sexual dysfunction, and incontinence, osteoporosis, glaucoma, and gastroparesis.

The compounds according to the invention can additionally be used for the treatment of primary and secondary Raynaud's phenomenon, of microcirculation impairments, claudication, peripheral and autonomic neuropathies, diabetic microangiopathies, diabetic retinopathy, diabetic ulcers on the extremities, CREST syndrome, erythematosis, onychomycosis, rheumatic disorders, and for promoting wound healing and skin tanning.

The compounds according to the invention are furthermore suitable for the treatment of acute and chronic pulmonary diseases such as respiratory distress syndromes (ALI, ARDS) and chronic obstructive airway disorders (COPD), and for treating acute and chronic renal failure.

The compounds described in the present invention also represent active ingredients for controlling central nervous system diseases characterized by disturbances of the NO/cGMP system. They are suitable in particular for improving perception, concentration, learning or memory after cognitive impairments like those occurring in particular in association with situations/diseases/syndromes such as mild cognitive impairment, age-associated learning and memory impairments, age-associated memory losses, vascular dementia, craniocerebral trauma, stroke, dementia occurring after strokes (post-stroke dementia), post-traumatic craniocerebral trauma, general concentration impairments, concentration impairments in children with learning and memory problems, Alzheimer's disease, Lewy body dementia, dementia with degeneration of the frontal lobes including Pick's syndrome, Parkinson's disease, progressive nuclear palsy, dementia with corticobasal degeneration, amyolateral sclerosis (ALS), Huntington's disease, multiple sclerosis, thalamic degeneration, Creutzfeld-Jacob dementia, HIV dementia, schizophrenia with dementia or Korsakoff's psychosis. They are also suitable for the treatment of central nervous system disorders such as states of anxiety, tension and depression, CNS-related sexual dysfunctions and sleep disorders, and for controlling pathological disturbances of the intake of food, stimulants and addictive substances.

The compounds according to the invention are furthermore also suitable for controlling cerebral blood flow and thus represent effective agents for controlling migraine. They are also suitable for the prophylaxis and control of the sequelae of cerebral infarctions such as stroke, cerebral ischemias and craniocerebral trauma. The compounds according to the invention can likewise be employed for controlling states of pain.

In addition, the compounds according to the invention have an anti-inflammatory effect and can therefore be employed as anti-inflammatory agents.

The present invention further relates to the use of the compounds according to the invention for the treatment and/or prevention of disorders, especially of the aforementioned disorders.

The present invention further relates to the use of the compounds according to the invention for producing a medicament for the treatment and/or prevention of disorders, especially of the aforementioned disorders.

The present invention further relates to a method for the treatment and/or prevention of disorders, especially of the aforementioned disorders, by using an effective amount of at least one of the compounds according to the invention.

The compounds according to the invention can be employed alone or, if required, in combination with other active ingredients. The present invention further relates to medicaments comprising at least one of the compounds according to the invention and one or more further active ingredients, in particular for the treatment and/or prevention of the aforementioned disorders. Examples of suitable combination active ingredients which may be preferably mentioned are:
organic nitrates and NO donors such as, for example, sodium nitroprusside, nitroglycerin, isosorbide mononitrate, isosorbide dinitrate, molsidomine or SIN-1, and inhaled NO;
compounds which inhibit the breakdown of cyclic guanosine monophosphate (cGMP), such as, for example, inhibitors of phosphodiesterases (PDE) 1, 2 and/or 5, in particular PDE 5 inhibitors such as sildenafil, vardenafil and tadalafil;
agents having antithrombotic activity, for example and preferably from the group of platelet aggregation inhibitors, of anticoagulants or of profibrinolytic substances;
active ingredients which lower blood pressure, for example and preferably from the group of calcium antagonists, angiotensin AII antagonists, ACE inhibitors, endothelin antagonists, renin inhibitors, alpha-receptor blockers, beta-receptor blockers, mineralocorticoid receptor antagonists, and of diuretics; and/or
active ingredients which modify lipid metabolism, for example and preferably from the group of thyroid receptor agonists, cholesterol synthesis inhibitors such as, for example and preferably, HMG-CoA reductase inhibitors or squalene synthesis inhibitors, of ACAT inhibitors, CETP inhibitors, MTP inhibitors, PPAR-alpha, PPAR-gamma and/or PPAR-delta agonists, cholesterol absorption inhibitors, lipase inhibitors, polymeric bile acid adsorbents, bile acid reabsorption inhibitors and lipoprotein (a) antagonists.

Agents having antithrombotic activity preferably mean compounds from the group of platelet aggregation inhibitors, of anticoagulants or of profibrinolytic substances.

In a preferred embodiment of the invention, the compounds according to the invention are administered in combination with a platelet aggregation inhibitor such as, for example and preferably, aspirin, clopidogrel, ticlopidine or dipyridamole.

In a preferred embodiment of the invention, the compounds according to the invention are administered in combination with a thrombin inhibitor such as, for example and preferably, ximelagatran, melagatran, bivalirudin or clexane.

In a preferred embodiment of the invention, the compounds according to the invention are administered in combination with a GPIIb/IIIa antagonist such as, for example and preferably, tirofiban or abciximab.

In a preferred embodiment of the invention, the compounds according to the invention are administered in combination with a factor Xa inhibitor such as, for example and preferably rivaroxaban (BAY 59-7939), DU-176b, apixaban, otamixaban, fidexaban, razaxaban, fondaparinux, idraparinux, PMD-3112, YM-150, KFA-1982, EMD-503982, MCM-17, MLN-1021, DX 9065a, DPC 906, JTV 803, SSR-126512 or SSR-128428.

In a preferred embodiment of the invention, the compounds according to the invention are administered in combination with heparin or with a low molecular weight (LMW) heparin derivative.

In a preferred embodiment of the invention, the compounds according to the invention are administered in combination with a vitamin K antagonist such as, for example and preferably, coumarin.

Agents which lower blood pressure preferably mean compounds from the group of calcium antagonists, angiotensin AII antagonists, ACE inhibitors, endothelin antagonists, renin inhibitors, alpha-receptor blockers, beta-receptor blockers, mineralocorticoid receptor antagonists, and of diuretics.

In a preferred embodiment of the invention, the compounds according to the invention are administered in combination with a calcium antagonist such as, for example and preferably, nifedipine, amlodipine, verapamil or diltiazem.

In a preferred embodiment of the invention, the compounds according to the invention are administered in combination with an alpha-1-receptor blocker such as, for example and preferably, prazosin.

In a preferred embodiment of the invention, the compounds according to the invention are administered in combination with a beta-receptor blocker such as, for example and preferably, propranolol, atenolol, timolol, pindolol, alprenolol, oxprenolol, penbutolol, bupranolol, metipranolol, nadolol, mepindolol, carazalol, sotalol, metoprolol, betaxolol, celiprolol, bisoprolol, carteolol, esmolol, labetalol, carvedilol, adaprolol, landiolol, nebivolol, epanolol or bucindolol.

In a preferred embodiment of the invention, the compounds according to the invention are administered in combination with an angiotensin AII antagonist such as, for example and preferably, losartan, candesartan, valsartan, telmisartan or embursatan.

In a preferred embodiment of the invention, the compounds according to the invention are administered in combination with an ACE inhibitor such as, for example and preferably, enalapril, captopril, lisinopril, ramipril, delapril, fosinopril, quinopril, perindopril or trandopril.

In a preferred embodiment of the invention, the compounds according to the invention are administered in combination with an endothelin antagonist such as, for example and preferably, bosentan, darusentan, ambrisentan or sitaxsentan.

In a preferred embodiment of the invention, the compounds according to the invention are administered in combination with a renin inhibitor such as, for example and preferably, aliskiren, SPP-600 or SPP-800.

In a preferred embodiment of the invention, the compounds according to the invention are administered in combination with a mineralocorticoid receptor antagonist such as, for example and preferably, spironolactone or eplerenone.

In a preferred embodiment of the invention, the compounds according to the invention are administered in combination with a diuretic such as, for example and preferably, furosemide.

Agents which modify lipid metabolism preferably mean compounds from the group of CETP inhibitors, thyroid receptor agonists, cholesterol synthesis inhibitors such as HMG-CoA reductase inhibitors or squalene synthesis inhibitors, of ACAT inhibitors, MTP inhibitors, PPAR-alpha, PPAR-gamma and/or PPAR-delta agonists, cholesterol absorption inhibitors, polymeric bile acid adsorbents, bile acid reabsorption inhibitors, lipase inhibitors and of lipoprotein(a) antagonists.

In a preferred embodiment of the invention, the compounds according to the invention are administered in combination with a CETP inhibitor such as, for example and preferably, torcetrapib (CP-529 414), JJT-705 or CETP vaccine (Avant).

In a preferred embodiment of the invention, the compounds according to the invention are administered in combination with a thyroid receptor agonist such as, for example and preferably, D-thyroxine, 3,5,3′-triiodothyronine (T3), CGS 23425 or axitirome (CGS 26214).

In a preferred embodiment of the invention, the compounds according to the invention are administered in combination with an HMG-CoA reductase inhibitor from the class of statins such as, for example and preferably, lovastatin, simvastatin, pravastatin, fluvastatin, atorvastatin, rosuvastatin, cerivastatin or pitavastatin.

In a preferred embodiment of the invention, the compounds according to the invention are administered in combination with a squalene synthesis inhibitor such as, for example and preferably, BMS-188494 or TAK-475.

In a preferred embodiment of the invention, the compounds according to the invention are administered in combination with an ACAT inhibitor such as, for example and preferably, avasimibe, melinamide, pactimibe, eflucimibe or SMP-797.

In a preferred embodiment of the invention, the compounds according to the invention are administered in combination with an MTP inhibitor such as, for example and preferably, implitapide, BMS-201038, R-103757 or JTT-130.

In a preferred embodiment of the invention, the compounds according to the invention are administered in combination with a PPAR-gamma agonist such as, for example and preferably, pioglitazone or rosiglitazone.

In a preferred embodiment of the invention, the compounds according to the invention are administered in combination with a PPAR-delta agonist such as, for example and preferably, GW 501516 or BAY 68-5042.

In a preferred embodiment of the invention, the compounds according to the invention are administered in combination with a cholesterol absorption inhibitor such as, for example and preferably, ezetimibe, tiqueside or pamaqueside.

In a preferred embodiment of the invention, the compounds according to the invention are administered in combination with a lipase inhibitor such as, for example and preferably, orlistat.

In a preferred embodiment of the invention, the compounds according to the invention are administered in combination with a polymeric bile acid adsorbent such as, for example and preferably, cholestyramine, colestipol, colesolvam, CholestaGel or colestimide.

In a preferred embodiment of the invention, the compounds according to the invention are administered in combination with a bile acid reabsorption inhibitor such as, for example and preferably, ASBT (=IBAT) inhibitors such as, for example, AZD-7806, S-8921, AK-105, BARI-1741, SC-435 or SC-635.

In a preferred embodiment of the invention, the compounds according to the invention are administered in combination with a lipoprotein (a) antagonist such as, for example and preferably, gemcabene calcium (CI-1027) or nicotinic acid.

The present invention further relates to medicaments which comprise at least one compound according to the invention, normally together with one or more inert, non-toxic, pharmaceutically suitable excipients, and to the use thereof for the aforementioned purposes.

The compounds according to the invention can act systemically and/or locally. For this purpose, they can be administered in a suitable way such as, for example, by the oral, parenteral, pulmonal, nasal, sublingual, lingual, buccal, rectal, dermal, transdermal, conjunctival, otic route or as implant or stent.

The compounds according to the invention can be administered in administration forms suitable for these administration routes.

Suitable for oral administration are administration forms which function according to the prior art and deliver the compounds according to the invention rapidly and/or in modified fashion, and which contain the compounds according to the invention in crystalline and/or amorphized and/or dissolved form, such as, for example, tablets (uncoated or coated tablets, for example having enteric coatings or coatings which are insoluble or dissolve with a delay and control the release of the compound according to the invention), tablets which disintegrate rapidly in the mouth, or films/wafers, films/lyophilisates, capsules (for example hard or soft gelatin capsules), sugar-coated tablets, granules, pellets, powders, emulsions, suspensions, aerosols or solutions.

Parenteral administration can take place with avoidance of an absorption step (e.g. intravenous, intraarterial, intracardiac, intraspinal or intralumbar) or with inclusion of an absorption (e.g. intramuscular, subcutaneous, intracutaneous, percutaneous or intraperitoneal). Administration forms suitable for parenteral administration are, inter alia, preparations for injection and infusion in the form of solutions, suspensions, emulsions, lyophilisates or sterile powders.

Suitable for the other administration routes are, for example, pharmaceutical forms for inhalation (inter alia powder inhalers, nebulizers), nasal drops, solutions or sprays; tablets for lingual, sublingual or buccal administration, films/wafers or capsules, suppositories, preparations for the ears or eyes, vaginal capsules, aqueous suspensions (lotions, shaking mixtures), lipophilic suspensions, ointments, creams, transdermal therapeutic systems (e.g. patches), milk, pastes, foams, dusting powders, implants or stents.

Oral or parenteral administration is preferred, especially oral administration.

The compounds according to the invention can be converted into the stated administration forms. This can take place in a manner known per se by mixing with inert, non-toxic, pharmaceutically suitable excipients. These excipients include, inter alia, carriers (for example microcrystalline cellulose, lactose, mannitol), solvents (e.g. liquid polyethylene glycols), emulsifiers and dispersants or wetting agents (for example sodium dodecyl sulfate, polyoxysorbitan oleate), binders (for example polyvinylpyrrolidone), synthetic and natural polymers (for example albumin), stabilizers (e.g. antioxidants such as, for example, ascorbic acid), colorants (e.g. inorganic pigments such as, for example, iron oxides) and masking flavors and/or odors.

It has generally proved advantageous to administer on parenteral administration amounts of about 0.001 to 1 mg/kg, preferably about 0.01 to 0.5 mg/kg, of body weight to achieve effective results, and on oral administration the dosage is about 0.01 to 100 mg/kg, preferably about 0.01 to 20 mg/kg, and very particularly preferably 0.1 to 10 mg/kg, of body weight.

It may nevertheless be necessary where appropriate to deviate from the stated amounts, in particular as a function of the body weight, route of administration, individual response to the active ingredient, nature of the preparation and time or interval over which administration takes place. Thus, it may be sufficient in some cases to make do with less than the aforementioned minimum amount, whereas in other cases the stated upper limit must be exceeded. It may in the event of administration of larger amounts be advisable to divide these into a plurality of individual doses over the day.

The following exemplary embodiments illustrate the invention. The invention is not restricted to the examples.

The percentage data in the following tests and examples are, unless indicated otherwise, percentages by weight; parts are parts by weight. Solvent ratios, dilution ratios and concentration data for the liquid/liquid solutions are in each case based on volume.

### A. EXAMPLES

### Abbreviations

aq. aqueous, aqueous solution
Bu butyl
cat. catalytic
DMF dimethylformamide
DMSO dimethyl sulfoxide
eq. equivalent(s)
ESI electrospray ionization (in MS)
Et ethyl
h hour(s)
HPLC high pressure, high performance liquid chromatography
LC/MS coupled liquid chromatography-mass spectrometry
LDA lithium diisopropylamide
Me methyl
min minute(s)
MS mass spectrometry
NMR nuclear magnetic resonance spectrometry
RT room temperature
Rₜ retention time (in HPLC)
THF tetrahydrofuran
UV ultraviolet spectrometry
v/v volume to volume ratio (of a solution)

### LC/MS and HPLC Methods:

### Method 1 (LC/MS):

Instrument: Micromass Quattro LCZ with HPLC Agilent series 1100; column: Phenomenex Synergi 2μ Hydro-RP Mercury 20 mm×4 mm; eluent A: 1 l water+0.5 ml 50% formic acid, eluent B: 1 l acetonitrile+0.5 ml 50% formic acid; gradient: 0.0 min 90% A→2.5 min 30% A→3.0 min 5% A→4.5 min 5% A; flow rate: 0.0 min 1 ml/min→2.5 min/3.0 min/4.5 min 2 ml/min; oven: 50° C.; UV detection: 208-400 nm.

### Method 2 (LC/MS):

MS instrument type: Micromass ZQ; HPLC instrument type: Waters Alliance 2795; column: Phenomenex Synergi 2μ Hydro-RP Mercury 20 mm×4 mm; eluent A: 1 l water+0.5 ml 50% formic acid, eluent B: 1 l acetonitrile+0.5 nil 50% formic acid; gradient: 0.0 min 90% A→min 30% A→3.0 min 5% A→4.5 min 5% A; flow rate: 0.0 min 1 ml/min→2.5 min/3.0 min/4.5 min 2 ml/min; oven: 50° C.; UV detection: 210 nm.

### Method 3 (LC/MS):

MS instrument type: Micromass ZQ; HPLC instrument type: HP 1100 series; UV DAD; column: Phenomenex Synergi 2μ Hydro-RP Mercury 20 mm×4 mm; eluent A: 1 l water+0.5 ml 50% formic acid, eluent B: 1 l acetonitrile+0.5 ml 50% formic acid; gradient: 0.0 min 90% A→2.5 min 30% A→3.0 min 5% A→4.5 min 5% A; flow rate: 0.0 min 1 ml/min→2.5 min/3.0 min/4.5 min 2 ml/min; oven: 50° C.; UV detection: 210 nm.

### Method 4 (LC/MS):

MS instrument type: Micromass ZQ; HPLC instrument type: HP 1100 series; UV DAD; column: Phenomenex Gemini 3μ 30 mm×3.00 mm; eluent A: 1 l water+0.5 ml 50% formic acid, eluent B: 1 l acetonitrile+0.5 ml 50% formic acid; gradient: 0.0 min 90% A→2.5 min 30% A→3.0 min 5% A→4.5 min 5% A; flow rate: 0.0 min 1 ml/min→2.5 min/3.0 min/4.5 min 2 ml/min; oven: 50° C.; UV detection: 210 nm.

### Method 5 (Preparative HPLC):

Column: Grom-Sil 120 ODS-4HE, 10 μm, 250 mm×30 mm; eluent A: 0.1% formic acid in water, eluent B: acetonitrile; flow rate: 50 ml/min; gradient: 0-3 min 10% B, 3-27 min 10% B→95% B, 27-34 min 95% B, 34-38 min 10% B.

### Method 6 (Preparative HPLC):

Column: Grom-Sil 120 ODS-4HE, 10 μm, 250 mm×30 mm; eluent A: water, eluent B: acetonitrile; flow rate: 50 ml/min; gradient A/B: 80:20→5:95.

### Starting Compounds and Intermediates

### Example 1A

### 1-(Cycloheptylmethyl)-1H-pyrazolo[3,4-b]pyridine-3-carbonitrile

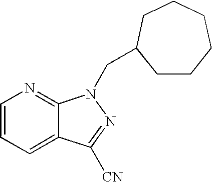

### Stage a)

### 3-(Trifluoromethyl)-1H-pyrazolo[3,4-b]pyridine

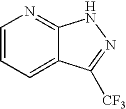

2-Fluoropyridine (2.00 g, 20.6 mmol) is added at −75° C. to a solution of freshly prepared LDA (22.7 mmol) in THF (60 ml). The solution is stirred at this temperature for 4 h. Then ethyl trifluoroacetate (18.4 g, 130 mmol) is rapidly added, during which the internal temperature rises to about 40° C. The mixture is cooled to −75° C. again, and then hydrazine hydrate (28.9 g, 577 mmol) is added. The reaction mixture is subsequently heated at 70° C. for 6 h. Volatile constituents are then removed in vacuo. Water (300 ml) is added to the residue, and the mixture is briefly brought to the boil while stirring vigorously. It is allowed to cool to RT and filtered with suction. The residue is taken up in ethyl acetate (300 ml), dried over sodium sulfate and clarified on activated carbon. Concentration results in 7.50 g (55% of theory) of the title compound as a slightly yellowish solid.

¹H-NMR (400 MHz, DMSO-d₆): δ=7.43 (dd, J=8.1, 4.4 Hz, 1H), 8.34 (d, J=8.1 Hz, 1H), 8.72 (dd, J=4.4, 1.5 Hz, 1H), 14.67 (br. s, 1H).

LC/MS (method 2): Rₜ=1.60 min.; MS (ESIpos): m/z=188 [M+H]⁺.

### Stage b)

### 1H-Pyrazolo[3,4-b]pyridine-3-carbonitrile

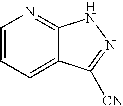

3-(Trifluoromethyl)-1H-pyrazolo[3,4-b]pyridine (500 mg, 2.67 mmol) is heated in 33% strength aqueous ammonia solution (10 ml) in a microwave at 140° C. for 10 min. The mixture is then concentrated in vacuo, and the residue is stirred with 100 ml of ethyl acetate and 20 ml of tert-butyl methyl ether at 70° C. Insoluble constituents are removed by suction filtration while hot, and the filtrate is concentrated. Drying results in 346 mg (90% of theory) of the title compound as pale beige crystals.

¹H-NMR (400 MHz, DMSO-d₆): δ=7.47 (dd, J=8.2, 4.5 Hz, 1H), 8.46 (dd, J=8.2, 1.5 Hz, 1H), 8.73 (dd, J=4.5, 1.5 Hz, 1H), 15.02 (br. s, 1H).

LC/MS (method 1): Rₜ=1.30 min.; MS (ESIpos): m/z=145 [M+H]⁺.

### Stage c)

### 1-(Cycloheptylmethyl)-1H-pyrazolo[3,4-b]pyridine-3-carbonitrile

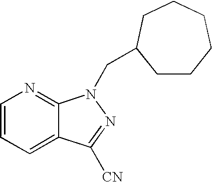

290 mg of 1H-pyrazolo[3,4-b]pyridine-3-carbonitrile (2.012 mmol) are dissolved in 5 ml of DMF, and 419 mg of cycloheptylmethyl methanesulfonate (2.012 mmol) and 656 mg of cesium carbonate (2.012 mmol) are added, and the mixture is stirred at room temperature for 16 h. A further 200 mg of cycloheptylmethyl methanesulfonate (0.969 mmol) and 320 mg cesium carbonate (0.982 mmol) are then added to the reaction mixture, which is stirred at room temperature for a further 2 days. A further 180 mg of cycloheptylmethyl methanesulfonate (0.872 mmol) and 282 mg of cesium carbonate (0.865 mmol) are added, and the reaction mixture is again stirred at room temperature for 2 days. Water is then added to the reaction mixture, and it is extracted three times with dichloromethane. The combined organic phases are washed with saturated sodium chloride solution, dried over sodium sulfate and concentrated in a rotary evaporator. The residue is purified by chromatography on silica gel (mobile phase:cyclohexane/ethyl acetate 5:1→2:1). 433 mg (85% of theory) of the target compound are obtained.

¹H-NMR (400 MHz, DMSO-d₆): δ=1.17-1.73 (m, 12H), 2.22-2.28 (m, 1H), 3.98 (d, J=6.4, 2H), 7.51 (dd, J=8.1, 4.4, 1H), 8.48 (dd, J=8.1, 1.5, 1H), 8.76-8.77 (m, 1H).

LC/MS (method 2): Rₜ=2.82 min.; MS (ESIpos): m/z=255 [M+H]⁺.

### Example 2A

### 1-(Cyclopentylmethyl)-1H-pyrazolo[3,4-b]pyridine-3-carbonitrile

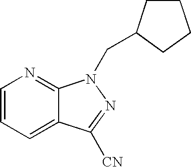

300 mg of 1H-pyrazolo[3,4-b]pyridine-3-carbonitrile (2.081 mmol; Example 1A, stage b) are dissolved in 10 ml of DMF, and 371 mg of cyclopentylmethyl methanesulfonate (2.081 mmol) and 678 mg of cesium carbonate (2.081 mmol) are added, and the mixture is stirred at room temperature for 16 h. Water is added to the reaction mixture, which is extracted three times with dichloromethane. The combined organic phases are washed with saturated aqueous sodium chloride solution and dried over sodium sulfate. The solvent is removed in a rotary evaporator, and the residue is purified by preparative HPLC (method 6). 9 mg (2% of theory) of the title compound are obtained.

LC/MS (method 2): Rₜ=2.48 min.; MS (ESIpos): m/z=227 [M+H]⁺.

### Example 3A

### 1-(Cyclohexylmethyl)-1H-pyrazolo[3,4-b]pyridine-3-carbonitrile

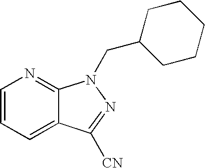

82 mg of 1H-pyrazolo[3,4-b]pyridine-3-carbonitrile (0.569 mmol; Example 1A, stage b) are dissolved in 3 ml of DMF, and 204 mg of cyclohexylmethyl bromide (1.138 mmol) and 371 mg of cesium carbonate (1.138 mmol) are added, and the mixture is stirred at room temperature for 24 h. Water is added to the reaction mixture, which is extracted three times with dichloromethane. The combined organic phases are washed with saturated aqueous sodium chloride solution and dried over sodium sulfate. The solvent is removed in a rotary evaporator, and the residue is purified by preparative HPLC (method 6). 26 mg (19% of theory) of the title compound are obtained.

¹H-NMR (400 MHz, DMSO-d₆): δ=0.97-1.23 (m, 5H), 1.47-1.66 (m, 5H), 1.95-2.06 (m, 1H), 4.46 (d, J=7.1, 2H), 7.51 (dd, J=8.3, 4.4, 1H), 8.48 (dd, J=8.3, 1.2, 1H), 8.76 (dd, J=4.4, 1.2, 1H).

LC/MS (method 2): Rₜ=2.63 min.; MS (ESIpos): m/z=241 [M+H]⁺.

### Example 4A

### 1-(2,3-Difluorobenzyl)-1H-pyrazolo[3,4-b]pyridine-3-carbonitrile

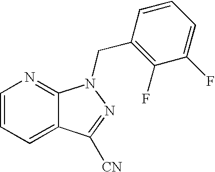

Cesium carbonate (244 mg, 0.75 mmol) is added to a solution of 1H-pyrazolo[3,4-b]pyridine-3-carbonitrile (90 mg, 0.62 mmol; Example 1A, stage b) and 2,3-difluorobenzyl bromide (142 mg, 0.69 mmol) in 1.8 ml of DMF under argon and at room temperature, and the reaction mixture is stirred for 16 h. For working up, 1.5 ml of 1 N hydrochloric acid and 3 ml of DMSO are added. The entire resulting solution is purified directly by preparative HPLC (method 5). 127 mg (75% of theory) of the title compound are obtained.

¹H-NMR (400 MHz, DMSO-d₆): δ=5.93 (s, 2H), 7.12-7.23 (m, 2H), 7.43 (dd, 1H), 7.55 (dd, 1H), 8.51 (dd, 1H), 8.81 (dd, 1H).

LC/MS (method 2): Rₜ=2.29 min.; MS (ESIpos): m/z=271 [M+H]⁺.

### Example 5A

### 1-(2,5-Difluorobenzyl)-1H-pyrazolo[3,4-b]pyridine-3-carbonitrile

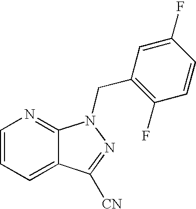

150 mg (80% of theory) of the title compound are obtained in analogy to Example 4A starting from 1H-pyrazolo[3,4-b]pyridine-3-carbonitrile (100 mg, 0.69 mmol) and 2,5-difluorobenzyl bromide (158 mg, 0.76 mmol).

¹H-NMR (400 MHz, DMSO-d₆): δ=5.88 (s, 2H), 7.20-7.35 (m, 3H), 7.56 (dd, 1H), 8.52 (dd, 1H), 8.81 (dd, 1H).

LC/MS (method 1): Rₜ=2.44 min.; MS (ESIpos): m/z=271 [M+H]⁺.

### Example 6A

### 1-(5-Chloro-2-fluorobenzyl)-1H-pyrazolo[3,4-b]pyridine-3-carbonitrile

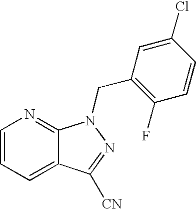

154 mg (70% of theory) of the title compound are obtained in analogy to Example 4A starting from 1H-pyrazolo[3,4-b]pyridine-3-carbonitrile (110 mg, 0.76 mmol) and 2-fluoro-5-chlorobenzyl bromide (188 mg, 0.84 mmol).

¹H-NMR (400 MHz, DMSO-d₆): δ=5.87 (s, 2H), 7.31 (dd, 1H), 7.46-7.51 (m, 2H), 7.55 (dd, 1H), 8.51 (dd, 1H), 8.81 (dd, 114

LC/MS (method 1): R₄=2.59 min.; MS (ESIpos): m/z=287 [M+H]⁺.

### Example 7A

### 1-[(5-Chloro-2-thienyl)methyl]-1H-pyrazolo[3,4-b]pyridine-3-carbonitrile

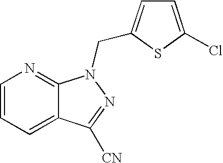

130 mg (74% of theory) of the title compound are obtained in analogy to Example 4A starting from 1H-pyrazolo[3,4-b]pyridine-3-carbonitrile (100 mg, 90% pure, 0.62 mmol) and 2-chloro-5-(chloromethyl)thiophene (125 mg, 0.75 mmol).

¹H-NMR (400 MHz, DMSO-d₆): δ=5.97 (s, 2H), 7.01 (d, 1H), 7.12 (d, 1H), 7.56 (dd, 1H), 8.51 (dd, 1H), 8.82 (dd, 1H).

### EXEMPLARY EMBODIMENTS

### Example 1

### 1-(Cycloheptylmethyl)-3-(1H-tetrazol-5-yl)-1H-pyrazolo[3,4-b]pyridine

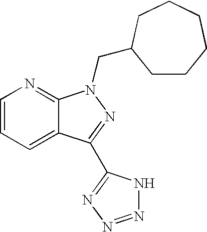

150 mg of the compound from Example 1A (0.590 mmol) are dissolved in 15 ml of toluene, and 140 mg of trimethylsilyl azide (1.180 mmol) and 15 mg of di-n-butyltin oxide (0.059 mmol) are added, and the mixture is heated under reflux for 24 h. After cooling, water is added to the reaction mixture, which is extracted three times with ethyl acetate. The combined organic phases are dried over sodium sulfate and concentrated in a rotary evaporator. The residue is purified by preparative HPLC (method 6). 43 mg (24% of theory) of the title compound are obtained.

¹H-NMR (400 MHz, DMSO-d₆): δ=1.23-1.40 (m, 4H), 1.43-1.65 (m, 8H), 2.28-2.35 (m, 1H), 4.46 (d, J=7.3, 2H), 7.48 (dd, J=8.1, 4.4, 1H), 8.68 (dd, J=8.1, 1.5, 1H), 8.73 (dd, J=4.4, 1.5, 1H), 17.31 (br. s, 1H).

LC/MS (method 2): Rₜ=2.25 min.; MS (ESIpos): m/z=298 [M+H]⁺.

### Example 2

### 1-(Cyclopentylmethyl)-3-(1H-tetrazol-5-yl)-1H-pyrazolo[3,4-b]pyridine

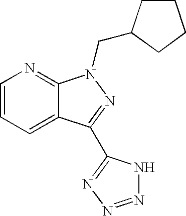

9 mg of the compound from Example 2A (0.039 mmol) are dissolved in 1 ml of toluene and 9 mg of trimethylsilyl azide (0.078 mmol) and 1 mg of di-n-butyltin oxide (0.004 mmol) are added, and the mixture is heated under reflux for 24 h. After cooling, water is added to the reaction mixture, which is extracted three times with ethyl acetate. The combined organic phases are dried over sodium sulfate and concentrated in a rotary evaporator. 10 mg (97% of theory) of the title compound are obtained.

LC/MS (method 3): Rₜ=2.19 min.; MS (ESIpos): m/z=270 [M+H]⁺.

### Example 3

### 1-(Cyclohexylmethyl)-3-(1H-tetrazol-5-yl)-1H-pyrazolo[3,4-b]pyridine

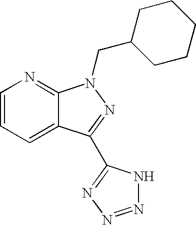

93 mg of the compound from Example 3A (0.386 mmol) are dissolved in 2 ml of toluene, and 99 mg of trimethylsilyl azide (0.772 mmol) and 10 mg of di-n-butyltin oxide (0.039 mmol) are added, and the mixture is heated under reflux for 16 h. After cooling, water is added to the reaction mixture, which is extracted three times with ethyl acetate. The combined organic phases are dried over sodium sulfate and concentrated in a rotary evaporator. The residue is purified by preparative HPLC (method 6). 90 mg (83% of theory) of the title compound are obtained.

¹H-NMR (400 MHz, DMSO-d₆): δ=1.02-1.20 (m, 5H), 1.52-1.67 (m, 5H), 2.02-2.11 (m, 1H), 4.47 (d, J=7.3, 2H), 7.47 (dd, J=8.1, 4.4, 1H), 8.68 (dd, J=8.1, 1.5, 1H), 8.72 (dd, J=4.4, 1.5, 1H), 17.30 (br. s, 1H).

LC/MS (method 1): Rₜ=2.30 min.; MS (ESIpos): m/z=284 [M+H]⁺.

### Example 4

### 1-(2,3-Difluorobenzyl)-3-(1H-tetrazol-5-yl)-1H-pyrazolo[3,4-b]pyridine

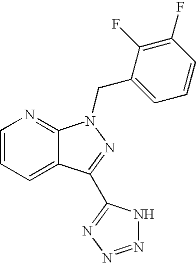

56 mg of 1-(2,3-difluorobenzyl)-1H-pyrazolo[3,4-b]pyridine-3-carbonitrile (0.21 mmol; Example 4A) are dissolved in 1.6 ml of toluene, and 48 mg of trimethylsilyl azide (0.41 mmol) and 5.1 mg of di-n-butyltin oxide (0.02 mmol) are added, and the mixture is heated under reflux overnight. After cooling, water is added to the reaction mixture, which is extracted three times with ethyl acetate. The combined organic phases are dried over sodium sulfate and concentrated in a rotary evaporator. The residue is purified by preparative HPLC (method 5). 63 mg (97% of theory) of the title compound are obtained.

¹H-NMR (400 MHz, CDCl₃): δ=5.96 (s, 2H), 7.06 (dd, 1H), 7.17 (dd, 1H), 7.41 (dd, 1H), 7.53 (dd, 1H), 8.72 (dd, 1H), 8.78 (dd, 1H), 17.38 (br. s, 1H).

LC/MS (method 2): Rₜ=1.88 min.; MS (ESIpos): m/z=314 [M+H]⁺.

### Example 5

### 1-(2,5-Difluorobenzyl)-3-(1H-tetrazol-5-yl)-1H-pyrazolo[3,4-b]pyridine

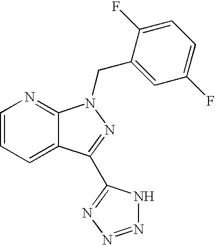

65 mg (75% of theory) of the title compound are obtained in analogy to Example 4 starting from 75 mg of 1-(2,5-difluorobenzyl)-1H-pyrazolo[3,4-b]pyridine-3-carbonitrile (0.28 mmol; Example 5A).

¹H-NMR (400 MHz, DMSO-d₆): δ=5.90 (s, 2H), 7.10-7.17 (m, 1H), 7.20-7.36 (m, 2H), 7.52 (dd, 1H), 8.72 (dd, 1H), 8.77 (dd, 1H).

LC/MS (method 1): Rₜ=2.03 min.; MS (ESIpos): m/z=314 [M+H]⁺.

### Example 6

### 1-(5-Chloro-2-fluorobenzyl)-3-(1H-tetrazol-5-yl)-1H-pyrazolo[3,4-b]pyridine

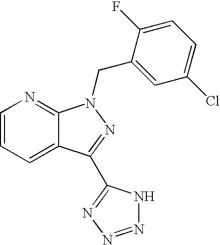

72 mg (98% of theory) of the title compound are obtained in analogy to Example 4 starting from 64 mg of 1-(5-chloro-2-fluorobenzyl)-1H-pyrazolo[3,4-b]pyridine-3-carbonitrile (0.22 mmol; Example 6A).

¹H-NMR (400 MHz, CDCl₃): δ=5.90 (s, 2H), 7.32 (dd, 1H), 7.41 (dd, 1H), 7.44-7.49 (m, 1H), 7.53 (dd, 1H), 8.72 (dd, 1H), 8.78 (dd, 1H), 17.35 (br. s, 1H).

LC/MS (method 2): Rₜ=1.99 min.; MS (ESIpos): m/z=330 [M+H]⁺.

### Example 7

### 1-[(5-Chloro-2-thienyl)methyl]-3-(1H-tetrazol-5-yl)-1H-pyrazolo[3,4-b]pyridine

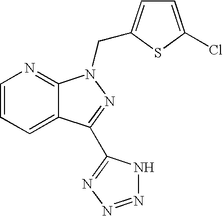

57 mg of 1-[(5-chloro-2-thienyl)methyl]-1H-pyrazolo[3,4-b]pyridine-3-carbonitrile (0.21 mmol; Example 7A), 5 mg of di-n-butyltin oxide (0.021 mmol) and 55 μl of trimethylsilyl azide (47.8 mg, 0.42 mmol) in 1.6 ml of toluene are stirred under reflux for 16 h. The reaction mixture is then cooled to room temperature, 1.6 ml of ethanol are added, and the mixture is stirred at room temperature for 1 h. For working up, 20 ml of ethyl acetate are added, and the solution is washed twice with 10 ml of water. The organic phase is dried over sodium sulfate and concentrated in a rotary evaporator. The residue is dissolved in DMSO and purified by preparative HPLC (method 5). 52 mg (79% of theory) of the title compound are obtained.

¹H-NMR (400 MHz, DMSO-d₆): δ=5.97 (s, 2H), 7.00 (d, 1H), 7.09 (d, 1H), 7.52 (dd, 1H), 8.71 (dd, 1H), 8.79 (dd, 1H).

LC/MS (method 4): Rₜ=2.51 min.; MS (ESIpos): m/z=318 [M+H]⁺.

### B. ASSESSMENT OF THE PHARMACOLOGICAL ACTIVITY

The pharmacological effect of the compounds of the invention can be shown in the following assays:

### B-1. Vasorelaxant Effect In Vitro

Rabbits are stunned by a blow to the back of the neck and are exsanguinated. The aorta is removed, freed of adherent tissue, divided into rings 1.5 mm wide and placed singly in 5 ml organ baths with carbogen-gassed Krebs-Henseleit solution of the following composition (in each case mM): NaCl: 119; KCl: 4.8; CaCl₂×2H₂O: 1; MgSO₄×7H₂O: 1.4; KH₂PO₄: 1.2; NaHCO₃: 25; glucose: 10, under an initial tension at 37° C. The force of contraction is detected with Statham UC2 cells, amplified and digitized via A/D converters (DAS-1802 HC, Keithley Instruments Munich), and recorded in parallel on chart recorders. A contraction is induced by adding phenylephrine to the bath cumulatively in increasing concentration. After several control cycles, the substance to be investigated is added in each further run in increasing dosage each time, and the level of contraction is compared with the level of contraction achieved in the last preceding run. The concentration necessary to reduce the level of contraction by 50% (IC₅₀) is calculated therefrom. The standard application volume is 5 μl and the DMSO content in the bath solution corresponds to 0.1%.

Representative IC₅₀ values for the compounds of the invention are shown in the table below:

| Example No. | IC₅₀ [μM] |
|---|---|
| 1 | 2.0 |
| 4 | 4.5 |
| 7 | 6.1 |

### B-2. Effect on Recombinant Guanylate Cyclase Reporter Cell Line

The cellular effect of the compounds of the invention is determined on a recombinant guanylate cyclase reporter cell line as described in F. Wunder et al., Anal. Biochem. 339, 104-112 (2005).

B-3. Determination of Pharmacokinetic Characteristics after Intravenous and Oral Administration

The substance to be investigated is administered to animals (e.g. mouse, rat, dog) intravenously as solution; oral administration takes place as solution or suspension by gavage. After administration of the substance, blood is taken from the animals at fixed times. This is heparinized and then plasma is obtained therefrom by centrifugation. The substance is quantified in the plasma analytically by LC/MS-MS. The pharmacokinetic characteristics such as AUC, Cₘₐₓ, T_{1/2} (half life) and CL (clearance) are calculated from the plasma concentration-time courses ascertained in this way, by means of a validated pharmacokinetic computer program.

### C. EXEMPLARY EMBODIMENTS OF PHARMACEUTICAL COMPOSITIONS

The compounds according to the invention can be converted into pharmaceutical preparations in the following ways:

### Tablet:

### Composition:

100 mg of the compound according to the invention, 50 mg of lactose (monohydrate), 50 mg of maize starch (native), 10 mg of polyvinylpyrrolidone (PVP 25) (from BASF, Ludwigshafen, Germany) and 2 mg of magnesium stearate.

Tablet weight 212 mg, diameter 8 mm, radius of curvature 12 mm

### Production:

A mixture of compound according to the invention, lactose and starch is granulated with a 5% strength solution (m/m) of the PVP in water. The granules are dried and subsequently mixed with the magnesium stearate for 5 minutes. This mixture is compressed in a conventional tablet press (see above for format of the tablet). A guideline compressive force for the compression is 15 kN.

Suspension which can be Administered Orally:

### Composition:

1000 mg of the compound according to the invention, 1000 mg of ethanol (96%), 400 mg of Rhodigel® (xanthan gum from FMC, Pennsylvania, USA) and 99 g of water.

10 ml of oral suspension correspond to a single dose of 100 mg of the compound according to the invention.

### Production:

The Rhodigel is suspended in ethanol, and the compound according to the invention is added to the suspension. The water is added while stirring. The mixture is stirred for about 6 h until the swelling of the Rhodigel is complete.

Solution which can be Administered Orally:

### Composition:

500 mg of the compound according to the invention, 2.5 g of polysorbate and 97 g of polyethylene glycol 400.20 g of oral solution correspond to a single dose of 100 mg of the compound according to the invention.

### Production:

The compound according to the invention is suspended in the mixture of polyethylene glycol and polysorbate with stirring. The stirring process is continued until the compound according to the invention has completely dissolved.

### i.v.-Solution:

The compound according to the invention is dissolved in a concentration below the saturation solubility in a physiologically tolerated solvent (e.g. isotonic saline, 5% glucose solution and/or 30% PEG 400 solution). The solution is sterilized by filtration and used to fill sterile and pyrogen-free injection containers.

1. A compound of the formula (I) 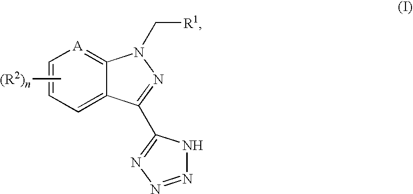
in which
A is CH, CR² or N,
R¹ is phenyl, pyridyl, furyl, thienyl, thiazolyl, oxazolyl, isothiazolyl or isoxazolyl, each of which may be substituted up to twice, identically or differently, by halogen, cyano, (C₁-C₄)-alkyl, trifluoromethyl and/or (C₂-C₄)-alkynyl,
or
is (C₅-C₇)-cycloalkyl which may be substituted up to twice, identically or differently, by fluorine and/or (C₁-C₄)-alkyl,
R² is a substituent selected from the series halogen, cyano, (C₁-C₄)-alkyl, trifluoromethyl, amino, (C₁-C₄)-alkoxy and trifluoromethoxy,
and
n is the number 0, 1 or 2,
where, in the event that the substituent R² occurs more than once, its meanings
may be identical or different,
and the salts, solvates and solvates of the salts thereof,
with the exception of the compounds 1-(2-fluorobenzyl)-3-(1H-tetrazol-5-yl)-1H-pyrazolo[3,4-b]pyridine, 1-(4-chlorobenzyl)-3-(1H-tetrazol-5-yl)-1H-indazole, 1-(2,4-dichlorobenzyl)-3-(1H-tetrazol-5-yl)-1H-indazole and 1-(4-chloro-2-methylbenzyl)-3-(1H-tetrazol-5-yl)-1H-indazole.

2. The compound of the formula (I) as claimed in claim 1, in which
A is N,
R¹ is pyridyl, furyl, thienyl, thiazolyl, oxazolyl, isothiazolyl or isoxazolyl, each of which may be substituted up to twice, identically or differently, by fluorine, chlorine, bromine, cyano, methyl and/or trifluoromethyl,
is (C₅-C₇)-cycloalkyl which may be substituted up to twice, identically or differently, by fluorine and/or methyl,
or
is a substituted ortho-fluorophenyl group of the formula 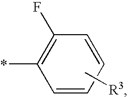
in which
* is the point of linkage
and
R³ is fluorine, chlorine, cyano, methyl or trifluoromethyl,
R² is a substituent selected from the series fluorine, chlorine, methyl, trifluoromethyl, amino, methoxy and trifluoromethoxy,
and
n is the number 0 or 1,
and the salts, solvates and solvates of the salts thereof.

3. The compound of formula (I) of claim 1, wherein the compound is one of the following structures: 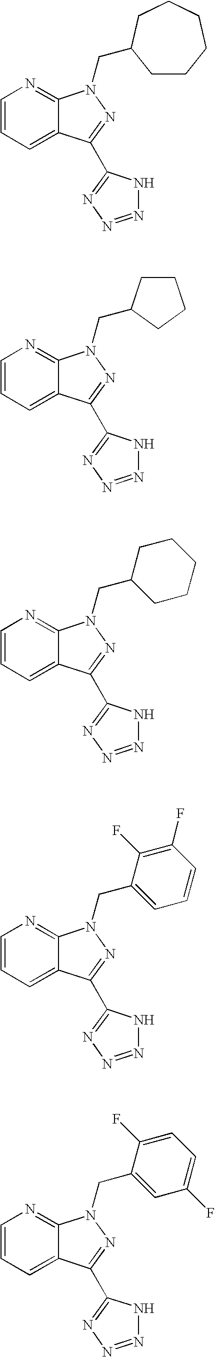 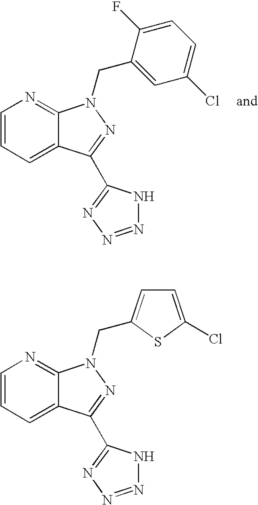
and the salts, solvates and solvates of the salts thereof.

4. A process for preparing a compound of formula (I) of claim 1, comprising converting a compound of formula (II) 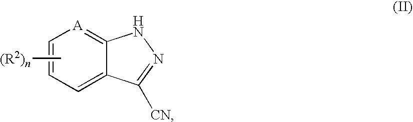
in which A, R² and n each have the meanings indicated in claim 1, in an inert solvent in the presence of a base with a compound of formula (III)
R¹—CH₂—X (III),
in which
R¹ has the meaning indicated in claim 1,
and
X is a leaving group such as halogen, mesylate, tosylate or triflate,
into a compound of the formula (IV) 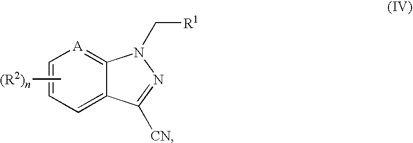
in which A, R¹, R² and n each have the meanings indicated above,
and the latter is then reacted in an inert solvent with an alkali metal azide in the presence of an acid or with trimethylsilyl azide, where appropriate in the presence of a catalyst, and the compounds of the invention obtained in this way are converted where appropriate with the appropriate (i) solvents and/or (ii) acids or bases into the solvates, salts and/or solvates of the salts thereof.

5. The compound of formula (I) of claim 1 for the treatment and/or prophylaxis of diseases.

6. (canceled)

7. A pharmaceutical composition comprising a compound of formula (I) of claim 1 in combination with an inert, non-toxic, pharmaceutically suitable excipient.

8. The pharmaceutical composition of claim 7, further comprising an active ingredient selected from the group consisting of an organic nitrate, an NO donor, a cGMP-PDE inhibitor, an agent having antithrombotic activity, an agent for lowering blood pressure, and an agent for altering lipid metabolism.

9. The pharmaceutical composition of claim 7 for the treatment and/or prevention of heart failure, angina pectoris, hypertension, pulmonary hypertension, ischemias, vascular disorders, thromboembolic disorders and arteriosclerosis.

10. A method for the treatment and/or prevention of heart failure, angina pectoris, hypertension, pulmonary hypertension, ischemias, vascular disorders, thromboembolic disorders and arteriosclerosis in humans and animals by administering a therapeutically effective amount of at least one compound of claim 1.

11. A method for the treatment and/or prevention of heart failure, angina pectoris, hypertension, pulmonary hypertension, ischemias, vascular disorders, thromboembolic disorders and arteriosclerosis in humans and animals by administering a therapeutically effective amount of a pharmaceutical composition of claim 7.
